(19) United States
(12) Patent Application Publication
(10) Pub. No.: US 2015/0051476 A1
(43) Pub. Date: Feb. 19, 2015

(54) METHOD FOR AN EVALUATION OF FIRST IMAGE DATA OF A FIRST IMAGING EXAMINATION AND SECOND IMAGE DATA OF A SECOND IMAGING EXAMINATION AND ALSO A MEDICAL IMAGING SYSTEM WHICH IS DESIGNED FOR CARRYING OUT THE METHOD

(71) Applicant: Siemens Aktiengesellschaft, Munich (DE)

(72) Inventors: Matthias FENCHEL, Erlangen (DE); Björn HEISMANN, Erlangen (DE); Kirstin JATTKE, Nuernberg (DE); Ralf LADEBECK, Erlangen (DE); Sebastian SCHMIDT, Weisendorf (DE); Harald WERTHNER, Fuerth (DE)

(21) Appl. No.: 14/451,689

(22) Filed: Aug. 5, 2014

(30) Foreign Application Priority Data
Aug. 15, 2013 (DE) 1020132162367

(51) Int. Cl. G06T 7/00; A61B 5/055; A61B 6/03; A61B 6/00

(52) U.S. Cl. CPC G06T 7/0026; A61B 6/4417; A61B 5/055; A61B 6/037; G06T 2207/10088; G06T 2207/10104; G06T 2207/30096
USPC 600/411; 382/131

(57) ABSTRACT

A method is disclosed for an evaluation of first image data of a first imaging examination and second image data of a second imaging examination on a patient. In an embodiment, the method includes a provision of first image data of the first imaging examination captured by way of a first imaging apparatus; a provision of second image data of the second imaging examination captured by way of a second imaging apparatus; a determination of first dynamic data on the basis of the first image data as a function of a time; a determination of second dynamic data on the basis of the second image data as a function of a time; and a determination of correlated data of a correlation of the first dynamic data with the second dynamic data.

### PRIORITY STATEMENT

The present application hereby claims priority under 35 U.S.C. §119 to German patent application number DE 102013216236.7 filed Aug. 15, 2013, the entire contents of which are hereby incorporated herein by reference.

### FIELD

At least one embodiment of the generally relates to a medical imaging system and method.

### BACKGROUND

In combined imaging systems, especially a combination of a magnetic resonance apparatus and a Positron Emission Tomography apparatus integrated within the magnetic resonance apparatus, the image data is captured by way of two technically different methods simultaneously or directly after each other. Two or more image datasets are captured here, which represent the same anatomical structures but have different image contents however, in respect of an image contrast or in respect of a voxel size for example.

In addition, in dynamic imaging, large volumes of data are captured by way of the respective imaging modality. However a correlation of the captured data is made more difficult because of the large volumes of data.

### SUMMARY

At least one embodiment of the present invention is directed to a simultaneous and dynamic data evaluation for magnetic resonance image data and Positron Emission Tomography image data. Advantageous embodiments are described in the dependent claims.

At least one embodiment of the invention is based on a method for an evaluation of first image data of a first imaging examination and second image data of a second imaging examination on the patient, comprising:
a provision of first image data of the first imaging examination captured by way of a first imaging apparatus,
a provision of second image data of the second imaging examination captured by way of a second imaging apparatus,
a determination of first dynamic data on the basis of the first image data as a function of a time,
a determination of second dynamic data on the basis of the second image data as a function of a time, and
a determination of correlated data of a correlation of the first dynamic data with the second dynamic data.

Furthermore, at least one embodiment of the invention is based on a medical imaging system with a first imaging apparatus, a second imaging apparatus and a processing unit, wherein the medical imaging system is designed for carrying out the method, for an evaluation of first image data of the first imaging examination and second image data of a second imaging examination on a patient, comprising:
a provision of first image data of the first imaging examination captured by way of a first imaging apparatus,
a provision of second image data of the second imaging examination captured by way of a second imaging apparatus,
a determination of first dynamic data on the basis of the first image data as a function of a time,
a determination of second dynamic data on the basis of the second image data as a function of a time, and
a determination of correlated data of a correlation of the first dynamic data with the second dynamic data.

At least one embodiment of the invention is further based on a computer program, which is able to be loaded directly into a memory of programmable processing unit of the medical imaging system, with program segments and/or modules to execute a method for an evaluation of first image data of the first imaging examination and second image data of a second imaging examination on a patient when the computer program is executed in the processing unit of the medical imaging system. A software-based realization of this type has the advantage that previous processing units of combined medical imaging systems which have a Positron Emission Tomography apparatus and a further imaging modality can be modified in a suitable way by implementation of the computer program.

### BRIEF DESCRIPTION OF THE DRAWINGS

Further advantages, features and details of the invention emerge from the exemplary embodiments described below and also with reference to the drawings.

In the figures:

FIG. 1 shows an inventive medical imaging system in a schematic diagram,

FIG. 2. shows a flowchart of an embodiment of an inventive method for an evaluation of first image data of a first imaging examination and second image data of a second imaging examination, and

FIG. 3a-c show a presentation of first dynamic data (FIG. 3a), second dynamic data (FIG. 3b) and correlated data determined from the first dynamic data and the second dynamic data (FIG. 3c).

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

At least one embodiment of the invention is based on a method for an evaluation of first image data of a first imaging examination and second image data of a second imaging examination on the patient, comprising:
a provision of first image data of the first imaging examination captured by way of a first imaging apparatus,
a provision of second image data of the second imaging examination captured by way of a second imaging apparatus,
a determination of first dynamic data on the basis of the first image data as a function of a time,
a determination of second dynamic data on the basis of the second image data as a function of a time, and
a determination of correlated data of a correlation of the first dynamic data with the second dynamic data.

At least one embodiment advantageously enables a relationship between first image data of the first imaging examination and second image data of the second imaging examination and/or a relationship between the first dynamic data and the second dynamic data to be determined and/or presented. In addition the individual dynamic data can be compared with each other and/or with the correlated data and additional specific and/or relevant information from the individual modality-specific data for the region of the body of the patient can be obtained therefrom.

In particular, the evaluations of the individual modality-specific image data captured no longer has to be undertaken separately, but rather the data can be correlated with one another and thus for example a tissue classification in the form of a correlation coefficient and/or specific perfusion characteristics of the examined area of the patient can be determined. For example individual tissue characteristics of the examined region of the body of a patient can be better captured and presented by the different imaging examinations, so that more specific information and/or findings can be obtained by a doctor on the basis of this dynamic data together with the correlated data. In addition there can be ongoing monitoring, in which especially ongoing changes of signal intensities are presented, by way of the dynamic data and/or the correlated data.

In this connection, a provision of image data is especially to be understood as loading of stored image data from a memory unit and/or a capture or an acquisition of raw image data by way of the first or second imaging apparatus. Furthermore dynamic data is especially to be understood as data which is preferably captured and/or acquired continuously over a period of time, preferably a period of time of the imaging examination, so that changes in the data values and/or signals during this time can be captured and/or presented. A correlation of the first dynamic data with the second dynamic data can for example include evaluations and/or algorithms which relate to different measured values of the different imaging examinations to one another and process the values. Model parameters can also be included in the calculation here.

An especially advantageous determination of correlated data of a correlation of the first dynamic data with the second dynamic data within the same period of time can advantageously be achieved if the underlying first image data and second image data for the determination of the correlated data is captured simultaneously. In particular in this way dynamic processes within the examined region of the body can be captured and presented at the same time by way of different examination modalities. Different information and/or knowledge which is produced as a result of different sensitivities of the different examination modalities just from the change of the first dynamic data of the second dynamic data for example, can be obtained in this way and, on the basis of a correlation of this dynamic data, a more comprehensive overall image of the examined area of the patient can be created. In this context a simultaneous capture of image data is especially to be understood as a simultaneous capture of the first and second image data.

Furthermore, it is proposed that the capture of the first image data and/or the capture of the second image data provided is undertaken during an administration of a medical device of indication or immediately after the administration of the medical device of indication. This advantageously enables a change of a metabolism and/or a change of an activity of the medical indication device and/or a change of a signal intensity of the medical indication device to be determined in a time-dependent manner and a characteristic and/or a parameter of the examined region to be derived therefrom. For example different perfusion characteristics of different medical indication devices which are captured by way of the different imaging apparatuses can be presented by way of a non-invasive monitoring.

In addition, it is proposed that the first image data comprises Positron Emission Tomography image data and the medical indication device is a radio pharmaceutical, through which advantageously characteristics, especially a size and/or a perfusion of tumor tissue and/or a tumor area can be captured. Preferably the radiopharmaceutical is 18-Fluorodeoxyglucose (FDG) or a 150-Tracer and/or further radiopharmaceuticals appearing sensible to the person skilled in the art, which are suitable for capture of dynamic Positron Emission Tomography image data.

In an advantageous development of at least one embodiment of the invention, it is proposed that the second image data comprises magnetic resonance image data, through which an especially advantageously high spatial resolution of the examined region of the body of the patient in the image data can be achieved. In combination with Positron Emission Tomography image data, an advantageous localization of the signals in the Positron Emission Tomography image data can also be achieved because of the high spatial resolution in the magnetic resonance image data. In addition an advantageous presentation of organs and soft tissue of patients can also be achieved by way of a magnetic resonance examination. The capture of the magnetic resonance image data for capturing and/or perfusion in the examined region of the body of the patient can be undertaken here during and/or directly after the administration of a medical indication device, especially of a magnetic resonance contrast device, such as for example Gd-DTPA (gadopentetate-dimeglumine). In addition a perfusion characteristic can also be produced by way of Arterial Spin Labeling (ASL).

Information about a distribution of medical indication device(s) and/or a period of time for which medical indication device(s) remain in the patient can be achieved especially simply if the first dynamic data and/or the second dynamic data include time-dependent signal values. Furthermore a perfusion analysis and thus an angiogenesis analysis, in which conclusions about tumor characteristics are obtained for example, such as a malignance, a tumor type, a local extent etc. are applied for example. Tumors are dependent on a capillary network which grows with them which supplies the tumor with oxygen and/or nutrients. Without the capability of being able to form new blood vessels for blood supply tumors remain restricted to a symptomless and clinically non-relevant size. Therefore precisely the information about a perfusion characteristic of the tumor tissue and/or of the examined region of the body is of particular interest for a further therapy planning and/or therapy checking. The time-dependent signal values can especially also include a time-dependent activity rate, as is especially sensible in the detection of Positron Emission Tomography image data.

In a further embodiment of the invention, it is proposed that pharmacokinetic models are used for determining the first dynamic data and/or for determining the second dynamic data. These enable an especially high expressiveness of the determined dynamic data to be achieved, since preferably metabolism characteristics and/or further process characteristics to which a medical indication device in the body of the patient is especially subjected, can be taken into account in the determination of the first dynamic data and/or the determination of the second dynamic data. In this context a pharmacokinetic model is especially to be understood as a model which especially includes a totality of all processes to which a medical indication device is subjected in the body of the patient. These processes can include a take-up of the medical indication device(s), a distribution of the medical indication device(s), a separation of the medical indication device(s) and also a biochemical breakdown and/or a biochemical conversion of the medical indication device(s).

Furthermore it is proposed that the first dynamic data and/or the second dynamic data and/or the correlated data is presented graphically, through which dynamic processes and/or a correlation between the different dynamic data of the examined area of the body of the patient can be presented especially simply and transferred to medical operating personnel, for example a doctor. In addition a non-invasive monitoring of the examined region of the body, especially a tumor region, of the patient can be achieved. Especially advantageously this can be achieved if the presentation of the first dynamic data and/or the presentation of the second dynamic data and/or the presentation of the correlated data is an ongoing process. Preferably the data is evaluated here to determine the first dynamic data and the second dynamic data simultaneously, so that where possible all relevant information is available for the ongoing monitoring. The ongoing monitoring preferably occurs over a limited time segment, especially the examination time for the medical imaging examination.

Furthermore, at least one embodiment of the invention is based on a medical imaging system with a first imaging apparatus, a second imaging apparatus and a processing unit, wherein the medical imaging system is designed for carrying out the method, for an evaluation of first image data of the first imaging examination and second image data of a second imaging examination on a patient, comprising:
a provision of first image data of the first imaging examination captured by way of a first imaging apparatus,
a provision of second image data of the second imaging examination captured by way of a second imaging apparatus,
a determination of first dynamic data on the basis of the first image data as a function of a time,
a determination of second dynamic data on the basis of the second image data as a function of a time, and
a determination of correlated data of a correlation of the first dynamic data with the second dynamic data.

Advantageously the individual dynamic data can be compared with each other and/or with the correlated data and from the comparison additional specific information from the individual modality-specific data of examined regions of the body of the patient can be obtained. For example, on the basis of the correlated dynamic data, a tissue classification can be determined in the form of a correlation coefficient and/or specific perfusion characteristics of the examined region of the patient can be determined.

At least one embodiment of the invention is further based on a computer program, which is able to be loaded directly into a memory of programmable processing unit of the medical imaging system, with program segments and/or modules to execute a method for an evaluation of first image data of the first imaging examination and second image data of a second imaging examination on a patient when the computer program is executed in the processing unit of the medical imaging system. A software-based realization of this type has the advantage that previous processing units of combined medical imaging systems which have a Positron Emission Tomography apparatus and a further imaging modality can be modified in a suitable way by implementation of the computer program.

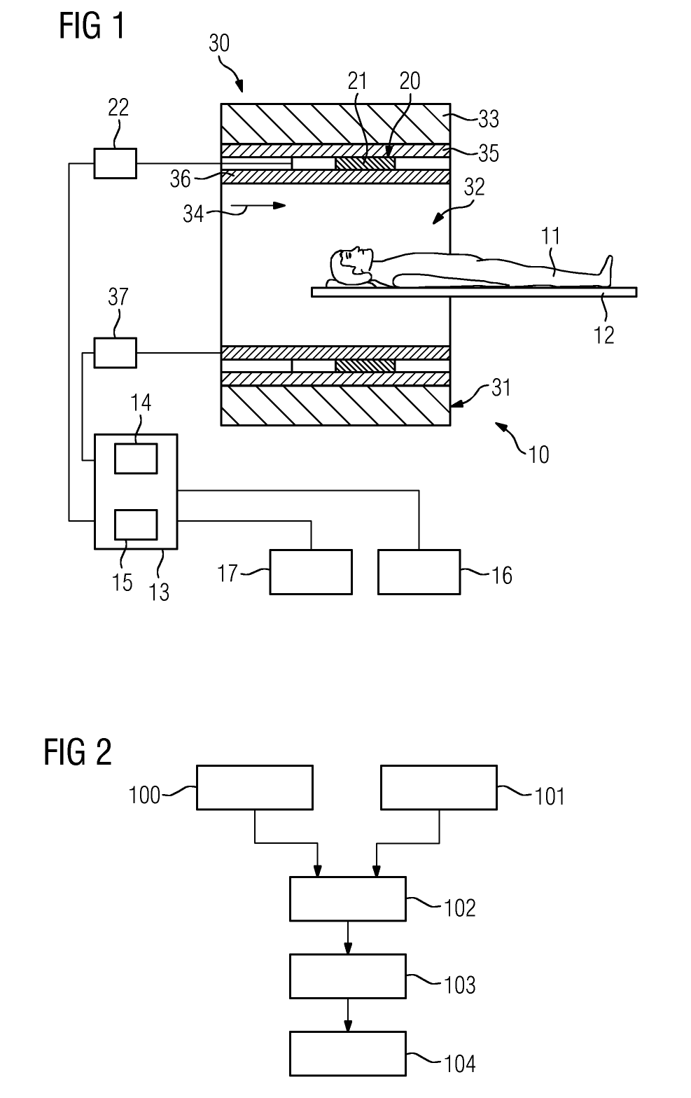

FIG. 1 shows a schematic of an embodiment of an inventive medical imaging system 10. The medical imaging system 10 comprises a combined imaging system which includes a first imaging apparatus and a second imaging apparatus. The first imaging apparatus is formed in the present example embodiment by a magnetic resonance apparatus 30. The second imaging apparatus is formed by a Positron Emission Tomography apparatus 20 (PET apparatus 20). Basically an embodiment of the first imaging apparatus as a computed tomography apparatus etc. is also conceivable, which is able to be combined with the PET apparatus 30.

The magnetic resonance apparatus 30 of the medical imaging system 10 comprises a magnet unit 31. The magnet unit 31 surrounds a patient receiving area 32 for capturing an image of a patient 11, wherein the patient receiving area 32 is surrounded in a circumferential direction by the magnet unit 31 in a cylindrical shape. The patient 11 can be pushed by way of the patient support apparatus 12 of the medical imaging system 10 into the patient receiving area 32. For this purpose the patient support apparatus 12 is disposed so as to enable it to be moved within the patient receiving area 32.

The magnet unit 31 comprises a main magnet 33, which is designed to create a strong and especially constant main magnetic field 34 during the operation of the magnetic resonance apparatus 30. The magnet unit 31 also has a gradient coil unit 35 for creating magnetic field gradients, which is used for local encoding during imaging. In addition the magnet unit 31 includes a high-frequency antenna unit 36 which is designed to excite a polarization which is generated in the main magnetic field 34 created by the main magnet 33.

To control the main magnet 33 of the gradient coil unit 35 and to control the high-frequency antenna unit 36 the magnetic resonance apparatus 30 has a control unit 37 formed by the processing unit. The control unit 37 centrally controls the magnetic resonance apparatus 30, such as for example the execution of a predetermined imaging gradient echo sequence. For this purpose the control unit 37 comprises a gradient control unit not shown in any greater detail and a high-frequency antenna control unit is not shown in any greater detail. In addition the control unit 37 comprises an evaluation unit not shown in any greater detail for evaluation of magnetic resonance image data.

The magnetic resonance image apparatus 30 shown can of course include further components which magnetic image apparatuses normally include. A general way in which a magnetic resonance apparatus 30 functions is also known to the person skilled in the art, so that a more detailed description of the general components will be dispensed with here.

The PET apparatus 20 of the medical imaging system 10 comprises a number of Positron Emission Tomography detector modules 21 (PET detector modules 21), which are disposed in the shape of a ring and surround the patient receiving area 32 in the circumferential direction. The PET detector modules 21 are disposed here between the high-frequency antenna unit 36 and the gradient coil unit 35 of the magnetic resonance apparatus 30 and are thus integrated into the magnetic resonance apparatus 30 in an especially space-saving way.

The PET detector modules 21 each have a number of Positron Emission Tomography detector elements (PET detector elements) not shown in any greater detail, which are arranged to form a PET detector array which comprises a scintillation detector array with scintillation crystals, for example LSO crystals. Furthermore the PET detector module 21 includes a photodiode array in each case, for example an avalanche photodiode array or APD photodiode array, which are disposed downstream from the scintillation detector array within the PET detector module 21. The PET detector array additionally has detector electronics, not shown in any greater detail, which includes an electric amplifier circuit and further electronic components not shown in any greater detail.

To control the PET detector module 21 the PET apparatus 20 has a control unit 22. The PET apparatus 20 shown can of course include further components which PET apparatuses normally include. A general way in which a PET apparatus 20 functions is also known to the person skilled in the art, so that a more detailed description of the general components will be dispensed with here.

Photon pairs which result from the annihilation of a positron with an electron are detected by way of the PET detector module 21. Trajectories of the two photons enclose an angle of 180°. In addition the two photons each have an energy of 511 keV. The positron here is emitted by a radiopharmaceutical, wherein the radiopharmaceutical is administered to the patient 11 via an injection. As they pass through material the photons arising during the annihilation can be absorbed, whereby the absorption probability depends on the path length through the material and the corresponding absorption coefficient of the material.

The medical imaging system 10 also has a central processing unit 13, which for example harmonizes a capture of magnetic resonance image data by way of the magnetic resonance apparatus 30 and the capture of PET image data by way of the PET apparatus 20 to one another for a joint data capture. The processing unit 13 also includes an evaluation unit not shown in any greater detail. The processing unit 13 further includes a processor unit 14 and a memory unit 15. Control information, such as imaging parameters for example and also reconstructed image data, can be displayed on a display unit 16, for example on at least one monitor, of the medical imaging system 10 for an operator. In addition the medical imaging system 10 has an input unit 17, by way of which information and/or parameters can be entered during a measurement process by an operator.

The medical imaging system 10 shown can of course include further components which medical imaging systems normally include. A general way in which a medical imaging system 10 functions is also known to the person skilled in the art, so that a more detailed description of the general components will be dispensed with here.

FIG. 2 shows a schematic flowchart of an embodiment of an inventive method for evaluating first image data of a first imaging examination and second image data of a second imaging examination on the patient 1.

In a first method step 100 first image data captured by way of the magnetic resonance apparatus which is formed by magnetic resonance image data of a region of the body of the patient 11 is provided by the processing unit 13 for a further evaluation. The provision of the magnetic resonance image data in the first method step 100 in this case includes a retrieval of already stored magnetic resonance image data from the memory unit 15 or also capturing and/or acquiring the magnetic resonance image data in the form of raw magnetic resonance image data by way of the magnetic resonance apparatus 30. In addition there can also be provision that the capture of the first magnetic resonance image data provided in the form of magnetic resonance raw image data to be undertaken during and/or after the administration of a medical indication device which is formed by a magnetic resonance contrast device, for example Gd-DTPA.

In a further method step 101, second image data captured by way of the PET apparatus 20, which is formed by a Positron Emission Tomography image data (PET image data) of the region of the body of the patient 11, is provided for a further evaluation by the processing unit 13. The region of the body of the patient 11 which is imaged by the PET image data is the same region of the body of the patient 11 which is imaged by the magnetic resonance image data. The provision of the PET image data in the further method step 101 in this case comprises a retrieval of already stored PET image data from the memory unit 15 or also capture and/or acquisition of the PET image data in the form of PET raw image data of the PET apparatus 20.

The capturing of the second image raw data provided by way of the PET apparatus 20 occurs during and/or after the administration of a medical indication device which is formed by a radio pharmaceutical. Preferably the radiopharmaceutical comprises 18-Fluorodeoxyglucose (FDG) or an 150-Tracer and/or further radiopharmaceutical is appearing sensible to the person skilled in the art which are suitable for the capture of dynamic Positron Emission Tomography image data. The capturing of the PET image data can begin up to 90 minutes after the administration of the radiopharmaceutical.

The magnetic resonance image data has a higher spatial resolution than the PET image data, so that by way of the magnetic resonance image data an especially unique assignment of PET signals in the PET image data to a point of origin in the human body is made possible during an evaluation of the PET image data.

The magnetic resonance image data provided and the PET image data provided are captured concurrently or simultaneously by way of the magnetic resonance imaging apparatus 30 and by way of the PET apparatus 20 respectively, so that dynamic processes in the examined region of the body of the patient 11 are captured and mapped simultaneously by way of the different imaging examination. Because of the simultaneous capturing of the magnetic resonance image data and the PET image data different information and/or knowledge about the examined region of the body is available, which is produced as a result of different sensitivities of the different examination modalities. As an alternative to this the magnetic resonance image data provided and the PET image data provided can also be captured with a short period of time between the two, wherein the short period of time between the capturing of the magnetic resonance image data and the PET image data has a time interval of maximum 15 minutes, advantageously of maximum ten minutes and especially preferred of maximum five minutes.

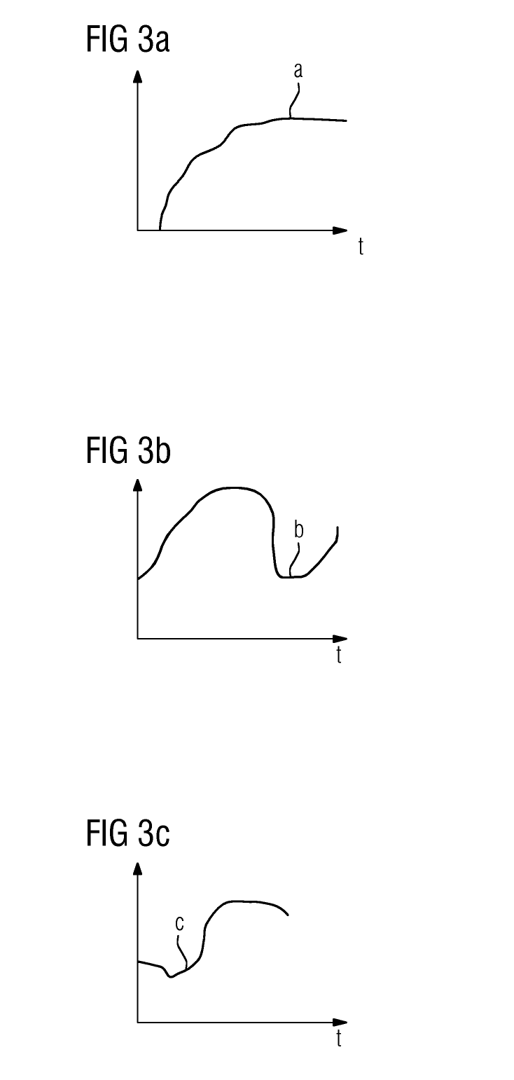

Subsequently first dynamic data is determined on the basis of the magnetic resonance image data by the processing unit 13 in a further method step 102 as a function of a time t (FIGS. 2 and 3a). In addition second dynamic data is likewise determined by the processing unit in the further method step 102 on the basis of the PET image data as a function of a time t (FIGS. 2 and 3b). The time t comprises an examination time of the first and/or the second imaging examination for example. In addition the time t can also include a dwell time of a medical indication device such as especially a radiopharmaceutical and/or the magnetic resonance contrast device. The first dynamic data and the second dynamic data is determined simultaneously by way of the processing unit 13 so that the first dynamic data and the second dynamic data are available for a common analysis.

The first dynamic data comprises time-dependent signal values a here, which in the present exemplary embodiment are formed by time-dependent magnetic resonance signals (FIG. 3a). The second dynamic data likewise comprise time-dependent signal values b, which are formed by a time-dependent activity rate and/or a change of an SUV (FIG. 3b). The SUV (standardized uptake value) comprises a physiological quantification of local concentrations of radioactivity. The SUV quantitatively includes a metabolism of the radiopharmaceutical used for the Positron Emission Tomography examination with the region of the body examined, for example a tumor and/or a lesion, of the patient 11. Here the SUV essentially represents a radioactivity concentration in relation to an applied radioactivity.

Furthermore, for determining the first and the second dynamic data, pharmacokinetic models are used by the processing unit 13. A correction of the PET image data and/or of the magnetic resonance image data is undertaken by the processing unit 13 by way of the pharmacokinetic model. The pharmacokinetic models allow information about a distribution and/or a concentration of the medical indication device, especially the radiopharmaceutical and/or the magnetic resonance contrast device. For example an arterial input function in the take-up of the radiopharmaceutical can be taken into consideration in the calculation and/or the determination of the change of signal intensity here.

In a further method step 103 correlated data of a correlation of the first dynamic data with the second dynamic data are determined by the processing unit 13 (FIGS. 2 and 3c). The correlated data is determined here by the processing unit in a time-dependent manner, so that for the correlated data a same capture time of the magnetic resonance image data and PET image data included in the calculation is present for the correlated data. On the basis of the correlated data in the further method step 103, a correlation coefficient can also be calculated by the processing unit 13, by way of which for example a tissue classification and/or specific perfusion characteristics of the examined region of the patient 11 are determined. The correlated data here includes a time-dependent correlation value c, such as a time-dependent correlation coefficient for example.

A correlation of the first dynamic data with the second dynamic data carried out by the processing unit 13 in the method step 103 can for example include evaluations and/or algorithms, which relate different measured values of the different imaging examinations to one another and process the values. Model parameters, for example from pharmacokinetic models, can also be included in the calculation here.

In a further method step 104 the first dynamic data, the second dynamic data and/or the correlated data are displayed graphically, wherein the graphic presentation is initiated by the processing unit. The first dynamic data, the second dynamic data and/or the correlated data are presented graphically by way of the display unit 16.

In addition the first dynamic data, the second dynamic data and the correlated data can be presented continuously in method step 104 by way of the display unit 16. This makes possible a continuous, non-invasive monitoring by way of the dynamic data and/or the correlated data of the examined region of the body of the patient 11.

By an embodiment of the inventive method individual tissue characteristics of the examined region of the body of the patient 11 are captured differently by the different imaging examinations or the different imaging apparatus and this information is made available to a doctor for a subsequent diagnosis on the basis of the provision and/or presentation of the first dynamic data, the second dynamic data and the correlated data. The presentations and/or provisions of the first dynamic data, the second dynamic data and the correlated data make it possible for a doctor, on the basis of this dynamic data together with the correlated data, to create more specific information and/or findings.

The method steps 100 to 104 are executed by the processing unit 13 together with the magnetic resonance apparatus 30 and the PET apparatus 20. For this purpose, processing unit 13 includes the necessary software and/or computer programs which are stored in the memory unit 15. The software and/or computer programs comprise program segments and/or modules which are designed to execute the method steps 100 to 104 of the described method for an evaluation of magnetic resonance imaging data of the first medical imaging examination and PET image data of the second imaging examination on a patient when the computer program and/or the software is executed in the processing unit 13 by way of the processor unit 14 of the medical imaging apparatus 10.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Although the invention has been illustrated and described in greater detail by the preferred example embodiment, the invention is not restricted by the disclosed examples and other variations can be derived therefrom by the person skilled in the art, without departing from the scope of protection of the invention.

What is claimed is:

1. A method for an evaluation of first image data of a first imaging examination and of second image data of a second imaging examination on a patient, the method comprising:
provisioning first image data of the first imaging examination, previously captured via a first imaging apparatus;
provisioning second data of the second imaging examination, previously captured via a second imaging apparatus;
determining first dynamic data on the basis of the first image data as a function of a time;
determining second dynamic data on the basis of the second image data as a function of a time; and
determining correlated data from a correlation of the first dynamic data with the second dynamic data.

2. The method of claim 1, wherein the first image data and second image data underlying the determining of the correlated data are captured concurrently.

3. The method of claim 1, wherein at least one of the first image data and the second image data is previously captured during an administration of a medical indication device or directly after the administration of a medical indication device.

4. The method of claim 3, wherein the first image data comprises Positron Emission Tomography image data and the medical indication device is a radiopharmaceutical.

5. The method of claim 1, wherein the second image data comprises magnetic resonance image data.

6. The method of claim 1, wherein at least one of the first dynamic data and the second dynamic data include a time-dependent signal value.

7. The method of claim 1, wherein pharmacokinetic models are used for determining at least one of the first dynamic data and the second dynamic data.

8. The method of claim 1, wherein at least one of the first dynamic data, the second dynamic data and the correlated data are presented graphically.

9. The method of claim 1, wherein at least one of the first dynamic data, the second dynamic data and the correlated data are presented continuously.

10. A medical imaging system, comprising:
a first imaging apparatus to capture first image data of the first imaging examination;
a second imaging apparatus to capture second image data of the second imaging examination; and
a processing unit to
determine first dynamic data on the basis of the first image data as a function of a time,
determine second dynamic data on the basis of the second image data as a function of a time, and
determine correlated data from a correlation of the first dynamic data with the second dynamic data.

11. A computer program, directly loadable into a memory of a programmable processing unit of a medical imaging system, comprising program segments for executing the method of claim 1, when the computer program is executed in the processing unit of the medical imaging system.

12. The method of claim 2, wherein at least one of the first image data and the second image data is previously captured during an administration of a medical indication device or directly after the administration of a medical indication device.

13. The method of claim 12, wherein the first image data comprises Positron Emission Tomography image data and the medical indication device is a radiopharmaceutical.

14. The method of claim 1, wherein the first image data comprises Positron Emission Tomography image data.

15. The method of claim 14, wherein the second image data comprises magnetic resonance image data.

16. The method of claim 2, wherein the first image data comprises Positron Emission Tomography image data.

17. The method of claim 16, wherein the second image data comprises magnetic resonance image data.

18. A computer program, directly loadable into a memory of a programmable processing unit of a medical imaging system, comprising program segments for executing the method of claim 2, when the computer program is executed in the processing unit of the medical imaging system.

19. A computer readable medium comprising program segments for executing, when the computer program is executed by a programmable processing unit of a medical imaging system, the method of claim 1.

20. A computer readable medium comprising program segments for executing, when the computer program is executed by a programmable processing unit of a medical imaging system, the method of claim 2.
